(19) United States
(12) Patent Application Publication
(10) Pub. No.: US 2014/0023688 A1
(43) Pub. Date: Jan. 23, 2014

(54) ENCAPSULATED OILS

(71) Applicants: Stephanie Budijono, Plainsboro, NJ (US); David L. Kaplan, Concord, MA (US); Valery Normand, East Windsor, NJ (US); Florenzo Omenetto, Wakefield, MA (US); Lahoussine Quali, Geneva (CH); Eleanor M. Pritchard, New Orleans, LA (US)

(72) Inventors: Stephanie Budijono, Plainsboro, NJ (US); David L. Kaplan, Concord, MA (US); Valery Normand, East Windsor, NJ (US); Florenzo Omenetto, Wakefield, MA (US); Lahoussine Quali, Geneva (CH); Eleanor M. Pritchard, New Orleans, LA (US)

(73) Assignee: Firmenich SA, Geneva (CH)

(21) Appl. No.: 13/940,671

(22) Filed: Jul. 12, 2013

Related U.S. Application Data
(60) Provisional application No. 61/786,279, filed on Mar. 14, 2013.
(60) Provisional application No. 61/671,336, filed on Jul. 13, 2012.

(51) Int. Cl. A61K 47/14; A23D 7/00; A61K 9/113; A61K 9/14; A61K 8/04

(52) U.S. Cl. CPC A61K 47/14; A61K 9/146; A61K 8/04; A61K 9/113; A23D 7/003
USPC 424/401; 424/499; 424/400; 426/602; 426/98

(57) ABSTRACT

Provided herein is a silk material compositions and methods for encapsulating and/or stabilizing oil, lipid, hydrophobic or lipophilic compounds including active agents in a silk matrix.

### CROSS-REFERENCE TO RELATED APPLICATION(S)

This application claims priority to United States Provisional Applications Nos.: 61/786,279 filed on Mar. 14, 2013 and 61/671,336 filed on Jul. 13, 2013.

### TECHNICAL FIELD

The technical field generally relates to compositions comprised of silk material and methods of using the silk material for encapsulating and/or stabilizing oil, lipid, hydrophobic or lipophilic compounds including active agents in a biocompatible matrix.

### BACKGROUND

Long used in the pharmaceutical industry to improve drug bioavailability, stabilize drugs against various degradation pathways, minimize side effects or modify drug release kinetics, encapsulation techniques have gained increased attention in other fields, particularly food (Gibbs et al., 1999; Madene et al., 2006) and fragrances (Berthier et al., 2010; Ouali et al., 2006). Lipids, though useful both as ingredients in food products and as solvents for hydrophobic substances, are generally difficult to disperse in aqueous media and can be susceptible to autooxidation (Gharsallaoui et al., 2007). Carotenoids in food products also suffer from susceptibility to degradation, which lowers the final nutritional properties of products (Gharsallaoui et al., 2007). Though sustained presence of volatile fragrances in consumer products is desirable because it is associated with a feeling of pleasantness or cleanliness, the volatility of fragrances prevents persistence over long time frames (Berthier et al., 2010; Ouali et al., 2006). Flavors can be the most valuable ingredients in food products, but these precious compounds are usually highly volatile and chemically unstable, degrading in the presence of air, light, moisture and high temperatures (Edris et al., 2001; Gouin, 2004; Baranauskienė et al., 2006; Madene et al., 2006; Kanakdande et al., 2007; Sohail et al., 2011). While various encapsulation materials and techniques have been previously reported (for reviews see Gouin, 2004; Gibbs et al., 1999; Gharsallaoui et al., 2007; Madene et al., 2006; Kuang et al., 2010), those encapsulation approaches require processing conditions which can degrade delicate compounds and/or compromise the food safety of the final product (such as exposure to high heat or the use of toxic cross-linking chemicals (Liu et al., 1996; Qian et al., 1997; Demura et al., 1989; Lu et al., 2010)). Accordingly, there is a strong need to develop techniques that can reduce the loss of volatile and/or lipophilic compounds and to sustain the presence of these compounds (e.g., fragrances, flavors) in consumer products and protect and stabilize, e.g., flavors, lipids and other sensitive food additives.

### SUMMARY

Microencapsulation is becoming increasingly important in the food, cosmetics and medicinal industries due to its potential to improve storage stability and delivery of volatile and delicate compounds, e.g., flavors and fragrances. The inventors have demonstrated novel foodsafe techniques for encapsulating oil in silk biomaterials using emulsion-based processes that exploit silk's unique properties, including amphiphilicity, biocompatibility, aqueous and ambient processing and tunable physical crosslinking behavior. In some embodiments, the sonication induced self-assembly of silk that has previously been applied to the fabrication of hydrogels was used in place of the thermal or chemical suspension crosslinking that is traditionally used to stabilize the aqueous protein phase in emulsions of water in oil (W/O) and oil in water in oil (O/W/O) type. Stable, physically silk-based material or a cross-linked silk micro- and macroparticles loaded with oil or loaded with water-soluble active agent were produced by sonicating silk solutions and aliquoting into an oil bath. Silk was stabilizing and oil microdroplets emulsified in aqueous silk solutions did not impede the self-assembly of silk into films or hydrogel networks.

The inventors have further demonstrated that, in O/W/O emulsions, particle morphology and the permeability of the silk to a lipophilic active agent in the interior phase were determined by silk solution concentration, processing and sonication. These oil encapsulating silk biomaterials can be used in various applications, e.g., delivery and stabilization of fragrances, flavors, lipids and oil soluble drugs.

Provided herein is a silk particle comprising at least two immiscible phases, a first immiscible phase comprising a silk-based material or a cross-linked silk matrix and a second immiscible phase comprising an active agent, wherein the first immiscible phase encapsulates the second immiscible phase and the second immiscible phase excludes a liposome.

Further provided herein is a composition comprising a plurality of lipid compartments encapsulated in a silk-based material or a cross-linked silk matrix.

Yet further provided herein is an oil in water emulsion comprising water, silk fibroin and an oil wherein the emulsion comprises a droplet comprising the oil encapsulated by the silk fibroin.

Further provided herein is a method of producing a silk particle comprising:
a. providing or obtaining an emulsion of non-aqueous droplets dispersed in a silk solution undergoing a sol-gel transition (where the silk solution remains in a mixable state); and
b. contacting a pre-determined volume of the emulsion with a non-aqueous phase, whereby the silk solution entraps at least one of the non-aqueous droplets and gels to form a silk particle dispersed in the non-aqueous phase.

Also provided herein is a method of delivering an active agent comprising applying or administering to a subject a composition comprising a silk-based material or a cross-linked silk matrix, the silk-based material or a cross-linked silk matrix encapsulating a lipid compartment with an active agent disposed therein, said silk-based material or a cross-linked silk matrix being permeable to the active agent such that the active agent is released through the silk-based material or a cross-linked silk matrix, at a pre-determined rate, upon application or administration of the composition to the subject.

Another method provided herein is a method comprising a step of: maintaining a composition, wherein the composition comprises at least one lipid compartment encapsulated a silk-based material or a cross-linked silk matrix and at least one active agent distributed in said at least one lipid compartment, and wherein the silk-based material or a cross-linked silk matrix is permeable to said at least one active agent such that the active agent is released through the silk-based material or a cross-linked silk matrix into an ambient surrounding at a pre-determined rate.

In another embodiment, provided herein is a oil dispersed in a matrix of silk fibroin.

In a further aspect, provided herein is an oil-in-water-in-oil emulsion comprising water, a first oil, a second oil and silk fibroin wherein the emulsion comprises a particle comprising the first oil wherein the particle is dispersed in a second oil.

In another embodiment, provided herein is a particle comprising a physically cross-linked silk fibroin and an oil.

Accordingly, provided herein relates to silk-based compositions encapsulating hydrophobic (e.g., oil) and/or lipophilic compounds, and methods of making the same. In some embodiments, a hydrophobic compound (e.g., oil), optionally loaded with a lipophilic compound, can be mixed with a silk solution to form oil droplets. In order to produce a stable emulsion of oil droplets in aqueous silk, the silk mixture can be subjected to sonication. In such embodiments, the sonicated silk solution containing a dispersion of oil droplets can be further introduced into an oil bath to form oil-loaded silk particles (e.g., silk particles encapsulating one or more oil droplets). Different embodiments of the composition described herein can be used, for example, in tissue engineering such as to model a tissue with high lipid content, or in controlled release and/or stabilization of a volatile or lipophilic agent such as flavors, fragrances, or drug molecules.

### BRIEF DESCRIPTION OF THE DRAWINGS

FIG. 1 show silk hydrogel formation following sonication represented schematically (Matsumoto et al. 2006; Wang et al., 2008b) accompanied by representative photographs of silk fibroin in the various stages in the gelation process.

FIG. 2 shows Emulsions of sunflower oil containing Oil Red O mixed with 7% (w/v) aqueous silk solution in a 1:3 (v/v) ratio of oil:silk, mixed with inversion (˜10 min) (A) prior to sonication and (B) after gentle sonication (10% amplitude for 5 seconds). Scale bars=250 μm.

FIG. 3 shows Casting oil-loaded silk films: (A) Microemulsion of limonene in silk solution; (B) TGA thermograms of silk films prepared from silk alone and limonene microemulsions in silk solution. Silk films prepared from (C) silk solution alone and (D) limonene microemulsion (1:3 oil:silk; silk is 6% (w/v) prepared with a 30 minute degumming time) cast using the same circular, Teflon-lined molds; Patterned silk films prepared from (E) silk solution alone and (F) oil microemulsion (1:20 oil in silk; silk is 3% (w/v) prepared with a 45 minute degumming time) cast using the same hologram-patterned mold.

FIG. 4 shows Sonicated silk solution held in spherical droplets in a sunflower oil bath (silk has not completed transition to hydrogel state, as evidenced by the slight translucence of the particles). (B) Sonicated silk solution containing a dispersion of Oil Red O loaded oil microdroplets held in spherical droplets in a sunflower oil bath. (C) Side view of sonicated silk solution containing green food coloring for ease of visualization (D) Hydrogel silk spheres prepared from sonicated silk alone allowed to complete crosslinking in a sunflower oil bath retain their shape after removal from the oil bath (E) Oil loaded silk hydrogel microspheres prior to dehydration (silk matrix is soft hydrogel) (F) oil loaded silk spheres characterized by a firmer, denser silk encapsulation matrix resulting from dehydration of the silk hydrogel network with overnight drying at ambient conditions.

FIG. 5 shows (A) Silk hydrogel macroparticles loaded with doxorubicin prepared by pipetting controlled volumes into sunflower oil bath. (B) Silk hydrogel macroparticles loaded with green food coloring prepared by pipetting controlled volumes (10 or 50 μL) into sunflower oil bath and dehydrated silk macroparticles prepared by drying silk hydrogel macroparticles (C-D) Silk microspheres prepared by sonication of silk into a sunflower oil bath (W/O emulsion) (silk contains 1:100 volumetric ratio blue food coloring for visualization). Scale bar=100 μL.

FIG. 6 is a Schematic of silk microparticle preparation using O/W/O emulsions containing sonicated aqueous silk fibroin solution as the encapsulating water phase. Once sonicated, silk begins transitioning to the physically cross-linked water-insoluble hydrogel state, but remains in solution state for controllable durations dependent on the silk properties and sonication parameters. In the solution state, oil can be emulsified in the silk solution, and the O/W emulsion can be further emulsified in a continuous oil phase. In the continuous oil phase, the oil-encapsulating silk droplets are held in a spherical conformation until crosslinking completes, at which point the silk becomes a stable, water-insoluble hydrogel encapsulation matrix for the oil.

FIG. 7 shows microparticles prepared using O/W/O emulsions with 60 minute degumming time regenerated silk fibroin solution. (A) O/W/O emulsion prepared with 6% (w/v) (higher concentration), 60 minute degumming time silk sonicated at an amplitude of 15% for 45 seconds (B) O/W/O emulsion prepared with 3% (w/v) (lower concentration), 60 minute degumming time silk sonicated at an amplitude of 15% for 30 seconds. Scale bars=300 μm.

FIG. 8 shows microparticles prepared using O/W/O emulsions with 6% (w/v) 30 minute degumming time regenerated silk fibroin solution treated with different sonication parameters: silk sonicated at an amplitude of 10% for 15 seconds (A-B) and silk sonicated at an amplitude of 15% for 15 seconds (C-D)

FIG. 9 shows absorbance at 518 nm (relative Oil Red O diffusion from internal oil capsule to external oil phase/sunflower oil bath) (A) No sonication (B) Silk solution of concentration of 3% (w/v) sonicated at 15% amplitude for 30 seconds, degumming duration of the silk is varied: 30 minutes or 60 minutes. (C) Silk solution of concentration of 6% (w/v) prepared using a 30 minute degumming duration exposed to varied sonication: no sonication, sonication at 10% amplitude for 15 seconds, or sonication at 15% amplitude for 15 seconds. (D) Silk solution of concentration of 6% (w/v) prepared using a 60 minute degumming duration exposed to varied sonication: no sonication, sonication at 15% amplitude for 30 seconds, or sonication at 15% amplitude for 45 seconds.

FIG. 10 shows formation of a silk “skin” in O/W/O microspheres: at the exterior oil-water interface the silk skin appears “baggy” (A) or forms “puckers” (B, white arrows).

FIG. 11 shows time-course of untreated, dye-loaded silk film dissolution in water. Untreated silk films loaded with indigo carmine (top row) and fluorescein (bottom row) begin dissolving within 3 minutes of exposure to 37° C. water and are fully dissolved after 30 minutes of immersion.

FIG. 12 shows hologram logo patterned silk films (a) top row is the mold used to generate the hologram, bottom row left side is a hologram-patterned silk film prepared from an oil microemulsion in silk (1:20 oil in silk; silk is 3% (w/v) prepared with a 45 minute degumming time), bottom right is a hologram-patterned silk film prepared from silk alone (b) another image of the hologram-patterned films prepared from silk solution alone and oil microemulsion in silk cast from the same mold showing the pattern more clearly (this prototype appears in the main body of the manuscript) FIG. 12. Hologram logo patterned silk films (a) top row is the mold used to generate the hologram, bottom row left side is a hologram-patterned silk film prepared from an oil microemulsion in silk (1:20 oil in silk; silk is 3% (w/v) prepared with a 45 minute degumming time), bottom right is a hologram-patterned silk film prepared from silk alone (b) another image of the hologram-patterned films prepared from silk solution alone and oil microemulsion in silk cast from the same mold showing the pattern more clearly (this prototype appears in the main body of the manuscript)

FIG. 13 shows (a) Hologram patterned patterned oil-loaded silk film prepared by casting the oil-silk microemulsion on plastic sheeting with an iridescent surface (b) close-up showing iridescence of the patterned silk film

FIG. 14 shows free-standing 2D micro-prism arrays prepared by casting oil-silk microemulsion on reflector-patterned silicone molds (a) without flash (b) with flash, demonstrating retention of reflector functionality.

FIG. 15 shows Silk hydrogel spheres prepared by sonicating the silk solution, and adding food coloring to the sonicated silk while still in the solution state (volume of food coloring added held constant, ratio of red, blue and yellow food coloring varied as noted), aliquoting into oil bath and allowing crosslinking to complete at ambient conditions of pressure and temperature

### DESCRIPTION

Microencapsulation of flavors and fragrances in carrier matrices has potential in the food and cosmetics industries because it can provide protection against degradation, prevent loss of volatile compounds, increase shelf-life and/or allow controlled release (Madene et al., 2006). Microencapsulation defines a process in which tiny particles or droplets are surrounded by a protective coating layer, or embedded within an encapsulating matrix or membrane, providing a physical barrier between the incorporated compound and the surrounding environment (Baranauskienė et al., 2006; Madene et al., 2006; Gharsallaoui et al., 2007; Sohail et al., 2011). Encapsulation protects sensitive fragrances and flavors from deterioration by shielding them from degradative conditions such as oxygen, moisture, temperature and light (Edris et al., 2001; Gouin, 2004; Kanakdande et al., 2007; Sohail et al., 2011). The barrier that the encapsulating material provides can also delay evaporation of volatile compounds (Jun-xia et al., 2011), particularly if the compound interacts with the encapsulant material (Gharsallaoui et al., 2007). Microencapsulation can also improve dispersion of oils and oil soluble ingredients (which would otherwise be immiscible) in aqueous environments (Kanakdande et al., 2007; Gharsallaoui et al., 2007). In addition to stabilizing and protecting encapsulated flavors, fragrances and oils, encapsulation may also be able to provide controlled release under desired conditions (Gharsallaoui et al., 2007). Controlled release may be defined as a method by which one or more active agents or ingredients are made available at a desired site and time and at a specific rate. This precise timing and targeting of release could be used to maximize a given compound's effectiveness and optimize dosage (Gouin, 2004; Ouali et al., 2006; Berthier et al., 2010; Kuang and Oliveira, 2010).

Various encapsulation materials and techniques have been previously reported (for reviews see Gouin, 2004; Gibbs et al., 1999; Gharsallaoui et al., 2007; Madene et al., 2006; Kuang et al., 2010). Requirements for materials used for encapsulation are summarized in Table 1.

TABLE 1
Criteria for Flavor and Fragrance Encapsulation Material

Yield highly stable encapsulated products
Tunable material properties to ensure appropriate release behavior
(rapid vs. sustained)
Effectively encapsulate without the use of crosslinking agents
Soluble in water
Controllable viscosity in aqueous solution
High emulsification activity (i.e. prevents lipid separation
from the emulsion during dehydration)
Process and materials should be relatively inexpensive
Formation of fine, dense network during matrix formation and drying
Safe for use in humans/edible for flavor encapsulations
Relatively high mechanical strength

Many biopolymers have been used for the microencapsulation of various food ingredients, including natural gums (gum arabic, alginates, carragenans), proteins (milk or whey proteins, gelatin), maltodextrins with different dextrose equivalences, and waxes and their blends (Gharsallaoui et al., 2007). Proteins are especially attractive encapsulant materials because their physicochemical properties (including amphiphilic character, ability to self-associate and interact with a variety of substances, high molecular weight, and molecular chain flexibility) provide excellent functional properties for encapsulation (including solubility, viscosity, emulsification and film-formation) (Madene et al., 2006; Gharsallaoui et al., 2007; Baranauskienė et al., 2006; Dickinson, 2011). During emulsion formation, protein molecules are able to act as emulsifiers by rapidly adsorbing at the newly formed oil-water interface, forming a steric-stabilizing layer (Arshady et al., 1990; Madene et al., 2006; Dickinson, 2011). Proteins also show high binding capacity for many flavor and fragrance compounds (Gharsallaoui et al., 2007; Baranauskienė et al., 2006). Gelatin is one of the most commonly used encapsulation materials, but suffers from serious drawbacks that limit its widespread use. Gelatin is highly viscous even in low concentrations, possesses low solubility in cold water, and gluteraldehyde (the chemical used to cross-link gelatin) is toxic to humans (Jun-xia et al., 2011). In addition, concerns regarding the safety of animal-derived proteins have increased in response to the recent emergence of diseases such as the prions (Chourpa et al., 2006)

Though many materials have been proposed for encapsulation in food, cosmetic and medicinal applications, silk fibroin is an especially attractive encapsulant material due to its unique array of chemical and physical properties. Silk fibroin is a biologically-derived protein polymer purified from the domesticated silkworm (Bombyx mori) cocoons that is FDA-approved, edible (Bayçin et al., 2007; Hanawa et al., 1995), non-toxic and relatively inexpensive (Qian et al., 1996). Silk exhibits excellent mechanical properties, biocompatibility (Leal-Egaña and Scheibel, 2010; Meinel et al., 2005; Panilaitis et al., 2003) and biodegrades to non-toxic products via proteolysis (Wang et al., 2008a; Horan et al., 2005). Fibroin has been widely applied to cosmetics, food and the chemical industry (Bayraktar et al., 2005) and has recently been investigated as a scaffold for tissue engineering (Wang et al., 2006, Altman et al., 2003) and a drug carrier for controlled release (Numata and Kaplan, 2010; Pritchard et al., 2011; Wenk et al., 2011).

While other encapsulation approaches require processing conditions which can potentially degrade delicate compounds and/or compromise the food safety of the final product (such as exposure to high heat or the use of toxic cross-linking chemicals (Liu et al., 1996; Qian et al., 1997; Demura et al., 1989; Lu et al., 2010)), stable silk biomaterials can be prepared using mild, ambient, aqueous processing conditions (Numata and Kaplan, 2010; Pritchard and Kaplan, 2011). In particular, silk self-assembly into films occurs during drying at ambient conditions of temperature and pressure (Hofmann et al., 2006) and physically cross-linked beta-sheet rich silk hydrogels have been prepared using sonication (Wang et al., 2008b).

Unlike many biologically derived proteins, silk is inherently stable to changes in temperature, pH and moisture (Kuzuhara et al., 1987; Omenetto and Kaplan, 2010) and is mechanically robust (Altman et al., 2003). Due to its unique block copolymer structure (consisting of large hydrophobic domains and small hydrophilic spacers), silk self-assembles into organized nanoscale crystalline domains (β-sheets) separated by more flexible hydrophilic spacers that produce a highly stabilizing environment for incorporated proteins and small molecules (Lu et al., 2009). For example, β-carotene is highly sensitive to oxidation after extraction (Desobry et al., 1997), but can be stabilized by adsorption onto silk fibroin due to the ability of silk to substitute for the proteins that naturally stabilize β-carotene in vivo (Ishii et al., 1995). The exceptional capacity of silk to stabilize sensitive incorporated compounds, including small molecules and proteins was previously discussed (Pritchard et al., 2012). Previously, a wide range of water-soluble compounds and proteins (including enzymes and growth factors) have been successfully encapsulated in silk biomaterials (Numata and Kaplan; Pritchard et al., 2011; Wenk et al., 2011; Pritchard et al., 2012). This stabilizing capacity represents a major advantage of silk, e.g., for sensitive compounds and oils prone to degradation. Despite the highly attractive applications of oil encapsulation (e.g., the stabilization and controlled release of volatile flavors and fragrances) and the unique properties of silk, fabrication approaches for oil loaded silk biomaterials have not been explored.

Silk delivery applications and systems may include such materials as films, Microsystems, 3D porous sponges, hydrogels and tubes.

Glue like proteins (sericins) removed from cocoon silk may be regenerated as a silk fibroin solution. Bombyx mori cocoons for example are boiled in alkaline solutions (sericin removal) to obtain a degummed silk fibroin (usable as fiber material). The degummed silk fibroin may be dissolved in an aqueous solution followed by dialysis to obtain an aqueous silk solution.

Formation of a silk “skin” in W/O microspheres may be formed and detected by microscopy: at the exterior oil-water interface the skin appears “baggy” or forms “puckers.”

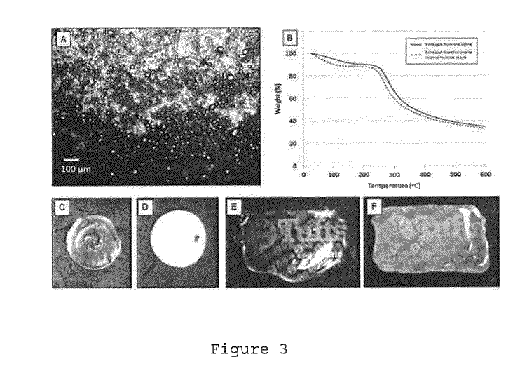

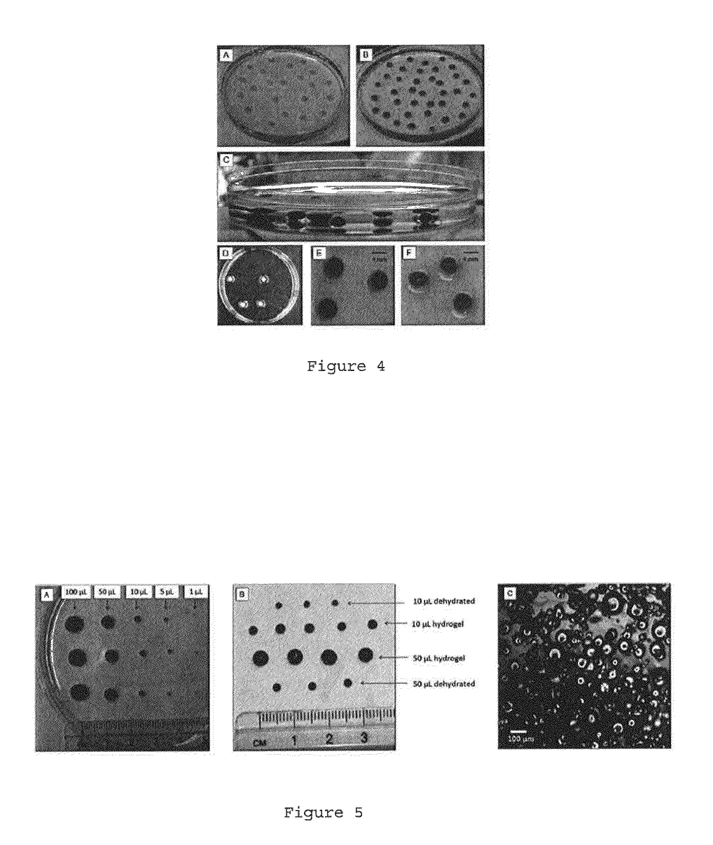

Further provided herein are W/O emulsions with physically cross-linked hydrogel microspheres. This may be carried out for example by sonicating a silk solution (e.g., 4% (w/v). These microspheres can then be added to an oil wherein the oil phase holds the silk droplet in a sphere while silk physically cross-links to form hydrogel microspheres. Microscale oil droplets produced by sonication are stabilized when silk protein is present in the continuous aqueous phase, and are maintained during self-assembly of silk films during drying (FIG. 3C-F) and self-assembly of silk hydrogel networks (FIG. 4B) following sonication, as expected based on the literature on proteins acting as emulsion stabilizers (Madene et al., 2006; Dickinson, 2011). Following dispersal of oil into the silk solution via sonication (FIG. 2A), this stable emulsion can be treated as silk solution and can be cast into films as previously described (Omenetto and Kaplan, 2010), rapidly-dissolving films (Kim et al., 2010), compound-loaded films for biosensors and diagnostics (Pritchard et al., 2012) and sustained release films for drug-delivery (Hofmann et al., 2006; Tsorias et al., 2012). TGA analysis revealed a slight decrease in thermostability of the silk films loaded with microparticles of oil compared with silk alone (FIG. 3B). However, self-assembly of the silk into films takes place on both Teflon coated molds (FIG. 3C-D) and patterned molds (FIG. 3E-F), even when the silk solution contained microparticles of oil.

Further provided herein is an O/W/O emulsion having cross-linked silk hydrogel microspheres. Sonication may be used to induce cross-linking Nanoscale droplets of oil are suspended in a soft silk hydrogel sphere. In one aspect, drying the physically cross-linked silk hydrogel network produces dense, dehydrated, spherical silk pellets.

Gentle, food-safe, aqueous methods for preparing oil-encapsulating silk biomaterials are described which can be used in food or pharmaceutical products particularly where protection, stabilization or controlled release are required. Many important chemotherapy drugs, steroids, hormones and antibiotics/antifungals are oil soluble but not highly water soluble and thus currently have to be administered with formulation additives like cremaphor or ethanol, which have side-effects in patients. In one embodiment, the inventors demonstrated encapsulation of sunflower oil, which represents the ability to encapsulate lipids alone (which can benefit from stabilization effects of encapsulation), but also models use of lipids as solvents in which hydrophobic substances such as volatile aromatic compounds (flavors and fragrances) and lipophilic vitamins and drugs could be solubilized for storage and delivery (Gharsallaoui et al., 2007). The encapsulation system described herein can be used in controlled release/drug delivery applications. Given the gentle, non-toxic, food-safe nature of the encapsulation process (e.g., films and spheres can be prepared at ambient conditions of temperature and pressure, stable emulsions produced without secondary emulsifiers or chemical cross-linking agents), the process described herein can be used for storage and delivery of flavors, fragrances, food additives, oils and oil-soluble compounds. Silk films prepared with oil in silk microemulsions can also be used for integrating oil-soluble diagnostic compounds like indicator dyes into diagnostic silk film based platforms that have been described previously.

In some embodiments, these products or silk-based compositions described herein can be used, for example, in pharmaceutical industry, food and consumer product industry, vendors that sell materials (fragrances, food additives or flavors) to the food and consumer product industry, producers of vitamins, supplements and probiotics; as well as in delivering nutritional supplements, vitamins, etc. to developing world settings where refrigeration is limited to address nutritional deficiencies.

In addition to having useful applications in food, cosmetics, consumer products and medicine, a stable dispersion of oil throughout a protein network can be more physiologically representative than a simple protein hydrogel in modeling tissues with high lipid content, such as the brain.

Emulsions are defined as mixtures of two immiscible phases (namely, water and oil) with an emulsifier added to stabilize the dispersed droplets (Dickinson, 2011). Emulsions are characterized as oil-in-water (O/W) or water-in-oil (W/O) depending on the identities of the dispersed and continuous phases. Multiple emulsions, such as oil-in-water-in-oil (O/W/O) emulsions, may be prepared to contain multiple phases. Previously, protein microspheres have been prepared from water-in-oil emulsions where aqueous protein solutions are dispersed in an oil bath (sometimes stabilized with emulsifiers and/or surfactants), then the proteins are stabilized via suspension crosslinking, either through thermal or chemical treatment (Arshady, 1990; Jayakrishnan et al., 1994; Esposito et al., 1996; Imsombut et al., 2010). Imsombut et al. have prepared silk microspheres using this method, with ethyl acetate as the oil phase, Span80 as an oil-soluble emulsifier, and genipin as a crosslinker (Imsombut et al., 2010). However, a process devoid of chemical additives is preferable as they can have toxic side-effects in vivo or damage delicate compounds (Esposito et al., 1996). Proteins droplets in water-in oil emulsions have been successfully converted to microparticles without chemical treatment by heating the oil bath to crosslink the protein matrix (Arshady, 1990; Esposito et al., 1996). However, heating is Particular to be avoided given the volatile nature of many fragrances and flavors (Jun-xia et al., 2011; Kanakdande et al., 2007). In contrast, sonication has been shown to induce the physical crosslinking of silk (Wang et al., 2008b). This mild process has been successfully applied to encapsulation of stem cells (Wang et al., 2008b) and labile proteins (Diab et al., in press).

Accordingly, sonication of silk, in some embodiments, is used for encapsulation of delicate, volatile compounds in oils. In addition, the sonication-induced silk gelation technique also possesses the advantage that the sol-gel transition time can be controlled through the sonication treatment. As a result, the silk remains in the solution state for tunable timeframes and compounds can be mixed into the silk solution prior to the final onset of gelation

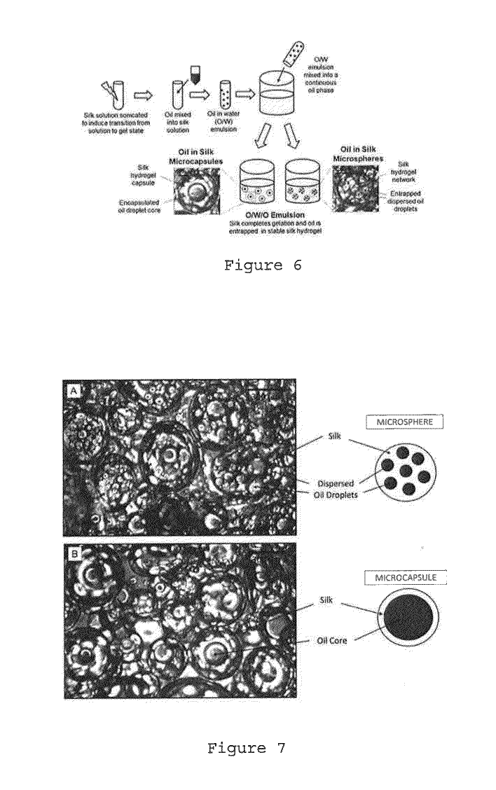

Silk microspheres comprising an oil may be formed by immersing empty silk microspheres in an oil solution allowing the oil to diffused into the empty microspheres. For example microspheres may be formed (See FIG. 6) wherein an agent or active ingredient is dispersed throughout a polymer matrix formed by the silk fibroin.

Films Prepared from Oil-in-Silk Microemulsions—Dissolution and Applications

Stable emulsion of microscale oil droplets dispersed in aqueous silk solution can be treated as silk solution and can be cast into films previously described (Omenetto and Kaplan, 2010), rapidly-dissolving films (Kim et al., 2010), compound-loaded films for biosensors and diagnostics (Pritchard et al., 2012) and sustained release films for drug-delivery (Hofmann et al., 2006; Tsorias et al., 2012).

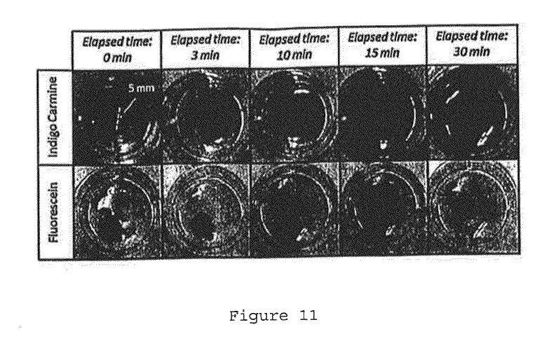

Previously, silk films that were cast and dried overnight at room temperature and ambient conditions that receive no additional treatment have been shown to dissolve rapidly upon exposure to an aqueous environment, such as immersion in buffer (shown below, FIG. 11) or when brought into contact with moist brain tissue as was recently shown with ultrathin electronics mounted onto dissolvable silk film substrates (Kim et al., 2010): these patterned films exhibited spontaneous conformal wrapping when applied to the soft, curvilinear surface of the brain tissue. Rapid dissolution and immobilized compound release of films loaded with dye is seen when the films are immersed in 37° C. buffer (FIG. 11). Dissolvable silk films loaded with 0.5, 0.25 or 0.125 mg of adenosine per 0.2 mm₂ film released the majority of the drug load (approx. 80%) within 15 minutes of exposure to 37° C. phosphate buffered saline (PBS).

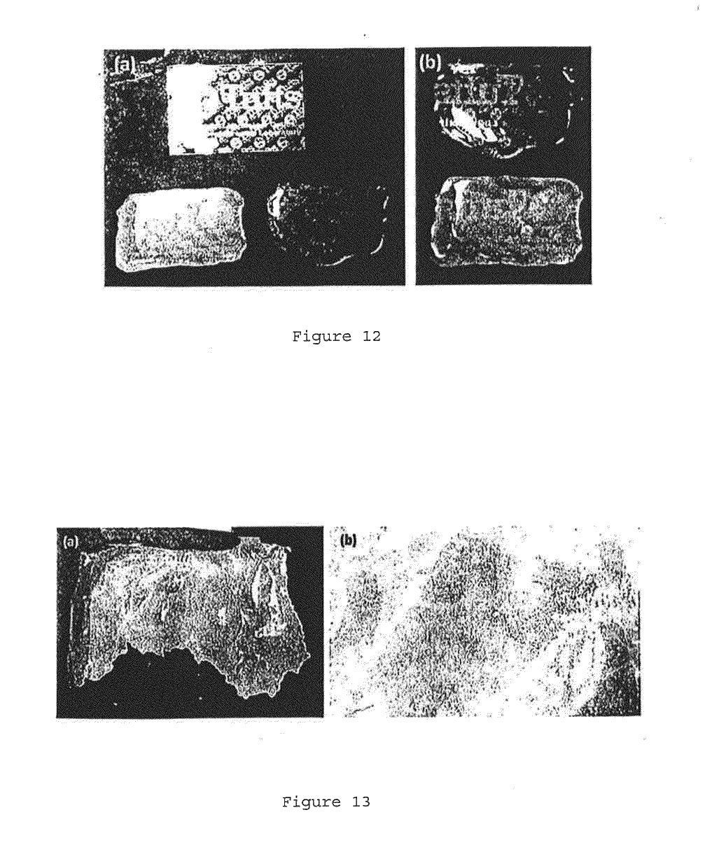

Oil-loaded silk films that were self-assembled by drying overnight at ambient conditions of temperature and pressure re-dissolved upon exposure to distilled water and phosphate buffered saline, releasing the incorporated oil and, presumably, any compounds carried in the oil (further quantification of this is needed). The capacity of water soluble silk films loaded with oil microdroplets to re-dissolve upon exposure to aqueous media indicates potential applications not only as a storage platform for oil-soluble therapeutics and nutrients, but also in the cosmetic and food industries, especially when combined with previously-explored patterning of silk substrates. For example, without wishing to be bound by theory, silk films containing microemulsions of flavor loaded oils can dissolve and release the encapsulated flavor once applied on the tongue or to the inside the cheek. Similarly, fragrance loaded untreated silk films re-dissolve if applied to slightly dampened skin Patterning of the silk films can further enhance the consumer's experience. Examples of patterned prototypes that we have demonstrated with microemulsions of fragrance loaded oils in silk are as follows (FIGS. 12-14):

Because the films can be treated post-drying to cross-link them, oil-soluble compounds with relevance to diagnostic devices can be integrated into previously-described silk platforms for these applications using this approach to film preparation.

### Hydrogel Silk Spheres (“Silk Pearls”)—Loading and Potential Applications

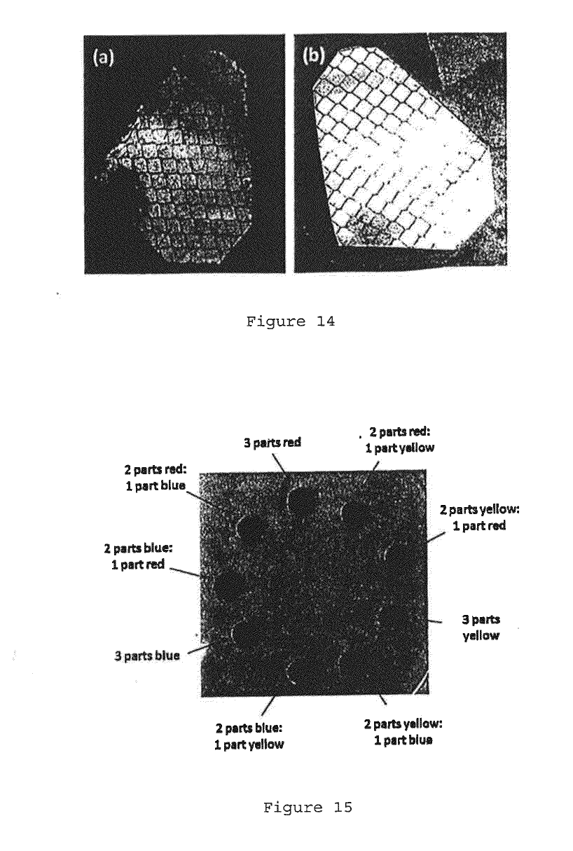

Tunable hydrogel silk spheres with controllable sizes or cross-linked “silk pearls” can be prepared from microemulsions of oil in silk or loaded with water soluble compounds. Controlling size/diameter of the spheres and potential post-cross-linking treatments can be used to extend functionality. For example, hydrogel silk pearls using varied ratios of food coloring demonstrates controlled loading of the spheres (FIG. 15). Because the preparation involves extrusion of the silk solution into oil baths and the volume and composition of the solution are controlled, encapsulation efficiency for non-oil-soluble compounds is theoretically 100% (unlike other microencapsulation approaches, where compound is frequently lost during processing). The high control and efficiency of loading is demonstrated by the food coloring loaded silk hydrogel sphere prototypes.

Because these silk hydrogel pearls are stable, but soft they have potential utility in food products comparable to tapioca pearls, bubble tea and vitamins (particularly oil-soluble/water insoluble vitamins and nutritional supplements such as fish oil, beta-carotene and vitamin E). Medication encapsulated in silk hydrogel pearls might represent a more easy to consume format for patients who have difficulty swallowing. Using silk instead of gelatin in food products and medication delivery formats offers the added advantage of alleviating the pathogen transmission concerns associated with use of mammalian sources. Because silk hydrogels are biocompatible and promote survival of encapsulated cells (Wang et al., 2008), these hydrogel pearls would be particularly useful for products containing probiotic bacteria: silk encapsulation might improve stability during storage (products with probiotics generally currently require refrigeration) and offer some degree of protection during exposure to the harsh environment of the stomach, improving the likelihood of the probiotic bacteria reaching their target site of action further along the GI tract.

In another embodiment, a microcapsule can also be formed (See FIG. 6) where a capsule core contains a volatile compound surrounded by a polymer membrane.

Provided herein is silk particle comprising at least two immiscible phases, a first immiscible phase comprising a silk based material or a cross-linked silk matrix and a second immiscible phase comprising an active agent, wherein the first immiscible phase encapsulates the second immiscible phase and the second immiscible phase excludes a liposome.

In another embodiment, the second immiscible phase comprises a lipid component.

In another embodiment, the lipid component comprises oil.

In another embodiment, the second immiscible phase forms a single compartment.

In another embodiment, the second immiscible phase forms a plurality of compartments.

In another embodiment, the size of the compartment or compartments ranges from about 1 μm to about 1000 μm, more particularly from about 10 μm to about 500 μm.

In another embodiment, the active agent present in the second immiscible phase comprises a hydrophobic or lipophilic molecule.

In another embodiment, the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a flavor, a fragrance or any combinations thereof.

In another embodiment, the cross-linked silk matrix comprises an additive.

In another embodiment, the additive comprises a biopolymer, an active agent, a plasmonic particle, glycerol, and any combinations thereof.

In another embodiment, a silk particle as described above is provided, wherein the second immiscible phase encapsulates a third immiscible phase.

In another embodiment, the cross-linked silk matrix is present in a form of a hydrogel.

In another embodiment, the cross-linked silk matrix is present in a dried state or lyophilized.

In another embodiment, a silk particle as described above, wherein the lyophilized silk matrix is porous.

In another embodiment, provided herein is a silk particle, wherein at least the cross-linked silk matrix in the first immiscible phase is soluble in an aqueous solution.

In another embodiment, provided herein is a silk particle, wherein beta-sheet content in the cross-linked silk matrix is adjusted to an amount sufficient to enable the cross-linked silk matrix to resist dissolution in an aqueous solution.

In another embodiment, the size of the silk particle ranges from about 0.1 mm to about 10 mm, more particularly from about 0.5 mm to about 5 mm.

In another embodiment, the exterior surface of the silk particle comprises less than 1% lipid component by weight.

Provided herein is a composition comprising a plurality of lipid compartments encapsulated in a cross-linked silk matrix.

In another embodiment, the lipid compartments ranges from about 1 μm to about 1000 μm, or from about 10 μm to about 500 μm.

In another embodiment, the volumetric ratio of the lipid compartments to the cross-linked silk matrix ranges from about 1:1 to about 1:1000, from about 1:5 to about 1:500, or from about 1: to about 1:100.

In another embodiment, the cross-linked silk matrix comprises a film.

In another embodiment, the cross-linked silk matrix comprises an optical pattern.

In another embodiment, the optical pattern comprises a hologram or an array of patterns that provides an optical functionality.

In another embodiment, the cross-linked silk matrix comprises a scaffold.

In another embodiment, lipid compartments further comprise an active agent.

In another embodiment, the active agent comprises a hydrophobic or lipophilic molecule.

In another embodiment, the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a flavor, fragrance or any combinations thereof.

In another embodiment, the cross-linked silk matrix comprises an additive.

In another embodiment, the additive comprises a biopolymer, an active agent, a plasmonic particle, glycerol, and any combinations thereof.

Further provided herein is a composition comprising a collection of silk particles.

In another embodiment, the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.

In another embodiment, the composition is formulated for use in a pharmaceutical product.

In another embodiment, the composition is formulated for use in a cosmetic product.

In another embodiment, the composition is formulated for use in a food product.

In another embodiment, the composition is a flavor.

In another embodiment, the composition is a fragrance.

In another embodiment provided herein is a storage-stable composition comprising a silk particle or a composition where the active agent present in the second immiscible phase of the silk particle, or a hydrophobic or lipophilic molecule present in the lipid components retains at least about 30% of its original bioactivity when the composition is (a) subjected to at least one freeze-thaw cycle, or (b) maintained for at least about 24 hours at a temperature of about room temperature or above, or (c) both (a) and (b).

In another embodiment, the composition is maintained under exposure to light.

In another embodiment, the composition is maintained at a relative humidity of at least about 10%.

In another embodiment, the cross-linked silk matrix is in a dried-state.

Provided herein is n oil in water emulsion comprising water, silk fibroin and an oil wherein the emulsion comprises a droplet comprising the oil encapsulated by the silk fibroin.

In another embodiment, the concentration of the silk fibroin is provided in an amount of about 6% (w/v) of the emulsion.

In another embodiment, the amount of the oil provided is in a ratio to the amount of silk fibroin provided of about 1:1 (v/v).

In another embodiment, the oil comprises a flavor or fragrance.

In another embodiment, the droplet has an average diameter of form about 2 micrometers up to about 1,000 micrometers

In another embodiment, the droplet has an average diameter of up to about 130 micrometers.

In another embodiment, the droplet has an average diameter of up to about 25 micrometers.

In another embodiment, the oil is provided in an amount of from about 10% to about 90%, by weight, of the total weight of the emulsion.

In another embodiment, the oil is provided in an amount of about 50% by weight of the total weight of the emulsion.

In another embodiment, the oil is a flavor or a fragrance.

Further provided herein is a film comprising an oil dispersed in a matrix of silk fibroin.

In another embodiment, the concentration of the silk fibroin is provided in an amount of about 6% (w/v) of the film.

In another embodiment, the amount of the oil provided is in a ratio to the amount of silk fibroin provided of about 1:1 (v/v).

In another embodiment, the oil is provided in an amount of from about 10% to about 90%, by weight, of the total weight of the film.

In another embodiment, the oil is provided in an amount of about 50% by weight of the total weight of the film.

In another embodiment, the oil in the film is a flavor or a fragrance.

Provided herein is an oil-in-water-in-oil emulsion comprising water, a first oil, a second oil and silk fibroin wherein the emulsion comprises a particle comprising the first oil wherein the particle is dispersed in a second oil.

In another embodiment, the emulsion comprises a physically cross-linked silk fibroin particle containing the first oil.

In another embodiment the particle comprises a macro-scale spherical particle.

In another embodiment, the particle comprises a micro-scale spherical particle.

In another embodiment, of the first oil is provided in an amount of from about 1 to 50%, by weight, of the total weight of the emulsion.

In another embodiment, the first oil is provided in an amount of about 15%, by weight, of the total weight of the solution.

In another embodiment, the first oil is a flavor or a fragrance.

In another embodiment, the particle is a microsphere comprising the first oil.

In another embodiment, the particle is a microcapsule comprising the first oil.

In another embodiment, the particle has an average diameter of about 2 micrometers up to about 5,000 micrometers.

Further provided herein is a particle comprising a physically cross-linked silk fibroin and an oil.

In another embodiment, the particle comprises a microsphere.

In another embodiment the amount of the oil is provided in an amount of about 10 to about 99%, by weight, of the total weight of the particle.

In another embodiment, the amount of the oil is provided in an amount of about 50%, by weight, of the total weight of the particle.

In another embodiment, the particle comprises a microcapsule

In another embodiment, the amount of the oil is provided in an amount of about 10 to about 99%, by weight, of the total weight of the particle.

In another embodiment, the amount of the oil is provided in an amount of about 80%, by weight, of the total weight of the particle.

In another embodiment, as the first oil is a flavor or a fragrance.

In another embodiment, the particle has an average diameter of about 2 micrometers up to about 1,000 micrometers.

Provided herein is a method of producing a silk particle comprising:
a. providing or obtaining an emulsion of non-aqueous droplets dispersed in a silk solution undergoing a sol-gel transition (where the silk solution remains in a mixable state); and
b. contacting a pre-determined volume of the emulsion with a non-aqueous phase, whereby the silk solution entraps at least one of the non-aqueous droplets and gels to form a silk particle dispersed in the non-aqueous phase.

In another embodiment, the sol-gel transition last for about at least 1 hour, or at least about 2 hours.

In another embodiment, the sol-gel transition of the silk solution is induced by sonication.

In another embodiment, the sonication is performed at an amplitude of about 5% to about 20%, or about 10% to about 15%.

In another embodiment, the sonication duration lasts for about 15 sec to about 60 sec, or from about 30 sec to about 45 sec.

In another embodiment, the silk solution has a concentration of about 1% (w/v) to about 15% (w/v), or about 2% (w/v) to about 7% (w/v).

In another embodiment, the method further comprises adding an active agent into the silk fibroin solution undergoing a sol-gel transition.

In another embodiment, the non-aqueous droplets further comprise a hydrophobic or lipophilic molecule.

In another embodiment, the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

In another embodiment, the emulsion is produced by adding a non-aqueous, immiscible phase into the silk solution, thereby forming the non-aqueous droplets dispersed in the silk solution.

In another embodiment, the pre-determined volume of the emulsion is a volume corresponding to a desirable size of the silk particle.

In another embodiment, the method further comprises isolating the silk particle from the non-aqueous phase.

In another embodiment, the method further comprises freeze-drying the silk particle.

Further provided herein is a method comprising a step of: maintaining a composition, wherein the composition comprises at least one lipid compartment encapsulated a cross-linked silk matrix and at least one active agent distributed in said at least one lipid compartment, and wherein the active agent retains at least about 30% of its original bioactivity when the composition is (a) subjected to at least one freeze-thaw cycle, or (b) maintained for at least about 24 hours at a temperature of about room temperature or above, or (c) both (a) and (b).

In another embodiment, the composition is maintained for at least about 1 month.

Provided further herein is a method comprising a step of: maintaining a composition, wherein the composition comprises at least one lipid compartment encapsulated a cross-linked silk matrix and at least one active agent distributed in said at least one lipid compartment, and wherein the cross-linked silk matrix is permeable to said at least one active agent such that the active agent is released through the cross-linked silk matrix into an ambient surrounding at a pre-determined rate.

In another embodiment, the pre-determined rate is controlled by adjusting an amount of beta-sheet conformation of silk fibroin present in the cross-linked silk matrix, porosity of the cross-linked silk matrix, or a combination thereof.

In another embodiment, the composition is maintained at about room temperature.

In another embodiment, the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.

In another embodiment, the composition is lyophilized.

In another embodiment, the composition is maintained at a temperature of about 37° C. or greater.

In another embodiment, the composition is maintained under exposure to light.

In another embodiment, the composition is maintained at a relative humidity of at least about 10%.

In another embodiment, of any of claims 52-61, the active agent comprises a hydrophobic or lipophilic active agent.

In another embodiment, the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a flavor, a fragrance or any combinations thereof.

In another embodiment, the cross-linked silk matrix comprises an additive.

In another embodiment, the additive comprises a biopolymer, an active agent, a plasmonic particle, glycerol, and any combinations thereof.

Provided here also is a method of delivering an active agent comprising applying or administering to a subject a composition comprising a cross-linked silk matrix, the cross-linked silk matrix encapsulating a lipid compartment with an active agent disposed therein, said cross-linked silk matrix being permeable to the active agent such that the active agent is released through the cross-linked silk matrix, at a pre-determined rate, upon application or administration of the composition to the subject.

In another embodiment, the active agent is released to an ambient surrounding.

In another embodiment, the active agent is released to at least one target cell of the subject.

In another embodiment, of the active agent comprises a hydrophobic or lipophilic active agent.

In another embodiment, the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a fragrance, a flavor, or any combinations thereof.

In another embodiment, the cross-linked silk matrix comprises an additive.

In another embodiment, the additive comprises a biopolymer, an active agent, a plasmonic particle, glycerol, and any combinations thereof.

In another embodiment, the composition is applied or administered to the subject topically or orally.

In another embodiment, the composition is applied on skin of the subject.

In some embodiments, the process described herein encompass mild processing options available for silk biomaterials (i.e., sonication-induced physical crosslinks, avoiding the use of cross-linking chemicals or heating) to produce encapsulation systems for flavors, fragrances, oils and oil-soluble compounds. For example, sunflower oil, as a model lipid, was encapsulated in silk to represent not only lipids alone (which can potentially benefit from stabilization effects of encapsulation), but also to model use of lipids as solvents in which hydrophobic substances such as volatile aromatic compounds (flavors and fragrances) and lipophilic vitamins and drugs could be solubilized for storage and delivery (Gharsallaoui et al., 2007).

Particular embodiments resort to the use of fragrance ingredients or compositions. By fragrance ingredients or composition it is understood here an ingredient or mixture of ingredients capable of activation an olfactive receptor of a human or animal subject. These are typically mixtures of ingredients capable of imparting a pleasant odor to the end product into which the microcapsules are incorporated, and the skilled perfumer is able to create such mixtures as a function of the perfuming effect that it is desired to impart and the perfumer's own creative capabilities.

The nature of the fragrance contained in the capsules is therefore immaterial in the context of the invention, provided that it is compatible with the materials forming the capsules. It will be typically chosen as a function of the perfuming effect that is desired to achieve with the dispersion or consumer product of the invention, and it will be formulated according to current practices in the art of perfumery. It may consist of a perfume ingredient or a composition. These terms can define a variety of odorant materials of both natural and synthetic origin, currently used for the preparation of perfumed consumer products. They include single compounds or mixtures. Specific examples of such components may be found in the current literature, e.g. Perfume and Flavor Chemicals by S. Arctander 1969, Montclair, N.J. (USA). These substances are well known to the person skilled in the art of perfuming consumer products, i.e. of imparting an odor to a consumer product traditionally fragranced, or of modifying the odor of said consumer product.

Natural extracts can also be encapsulated into the system of the invention; these include e.g. citrus extracts such as lemon, orange, lime, grapefruit or mandarin oils, or essentials oils of plants, herbs and fruits, amongst other.

Particular ingredients are those having a high steric hindrance and in particular those from one of the following groups:
Group 1: perfuming ingredients comprising a cyclohexyl, cyclohexenyl, cyclohexanone or cyclohexenone ring substituted with at least one linear or branched C₁ to C₄ alkyl or alkenyl substituent;
Group 2: perfuming ingredients comprising a cyclopentyl, cyclopentenyl, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexyl, cyclohexenyl, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C₇ to C₂₀ ring structure;
Group 7: perfuming ingredients having a logP value above 3.5 and comprising at least one tert-butyl or at least one trichloromethyl substitutent.

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, Romascone® (methyl 2,2-dimethyl-6-methylene-1-cyclohexanecarboxylate, origin: Firmenich SA, Geneva, Switzerland), nerone, terpineol, dihydroterpineol, terpenyl acetate, dihydroterpenyl acetate, dipentene, eucalyptol, hexylate, rose oxide, Perycorolle® ((S)-1,8-p-menthadiene-7-ol, origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, Doremox® (tetrahydro-4-methyl-2-phenyl-2H-pyran, origin: Firmenich SA, Geneva, Switzerland), cyclohexyl acetate, cyclanol acetate, Fructalate (1,4-cyclohexane diethyldicarboxylate, origin: Firmenich SA, Geneva, Switzerland), Koumalactone® ((3ARS,6SR,7ASR)-perhydro-3,6-dimethyl-benzo[B]furan-2-one, origin: Firmenich SA, Geneva, Switzerland), Natactone ((6R)-perhydro-3,6-dimethyl-benzo[B]furan-2-one, origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), Polysantol® ((1′R,E)-3,3-dimethyl-5-(2′,2′,3′-trimethyl-3′-cyclopenten-1′-yl)-4-penten-2-ol, origin: Firmenich SA, Geneva, Switzerland), fleuramone, Paradisone® (methyl-(1R)-cis-3-oxo-2-pentyl-1-cyclopentane acetate, origin: Firmenich SA, Geneva, Switzerland), Veloutone (2,2,5-Trimethyl-5-pentyl-1-cyclopentanone, origin: Firmenich SA, Geneva, Switzerland), Nirvanol® (3,3-dimethyl-5-(2,2,3-trimethyl-3-cyclopenten-1-yl)-4-penten-2-ol, origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, Neobutenone® (1-(5,5-dimethyl-1-cyclohexen-1-yl)-4-penten-1-one, origin: Firmenich SA, Geneva, Switzerland), nectalactone ((1′R)-2-[2-(4′-methyl-3′-cyclohexen-1′-yl)propyl]cyclopentanone), alpha-ionone, beta-ionone, damascenone, Dynascone® (mixture of 1-(5,5-dimethyl-1-cyclohexen-1-yl)-4-penten-1-one and 1-(3,3-dimethyl-1-cyclohexen-1-yl)-4-penten-1-one, origin: Firmenich SA, Geneva, Switzerland), Dorinone® beta (1-(2,6,6-trimethyl-1-cyclohexen-1-yl)-2-buten-1-one, origin: Firmenich SA, Geneva, Switzerland), Romandolide® ((1S,1′R)-[1-(3′,3′-Dimethyl-1′-cyclohexyl)ethoxycarbonyl]methyl propanoate, origin: Firmenich SA, Geneva, Switzerland), 2-tert-butyl-1-cyclohexyl acetate (origin: International Flavors and Fragrances, USA), Limbanol® (1-(2,2,3,6-tetramethyl-cyclohexyl)-3-hexanol, origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, Lorysia® (4-(1,1-dimethylethyl)-1-cyclohexyl acetate, origin: Firmenich SA, Geneva, Switzerland), 8-methoxy-1-p-menthene, Helvetolide® ((1S,1′R)-2-[1-(3′,3′-dimethyl-1′-cyclohexyl)ethoxy]-2-methylpropyl propanoate, origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate;
Group 4: Methyl cedryl ketone (origin: International Flavors and Fragrances, USA), Verdylate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), Hivernal® (a mixture of 3-(3,3-dimethyl-5-indanyl)propanal and 3-(1,1-dimethyl-5-indanyl)propanal, origin: Firmenich SA, Geneva, Switzerland), Rhubofix® (3′,4-dimethyl-tricyclo[6.2.1.0(2,7)]undec-4-ene-9-spiro-2′-oxirane, origin: Firmenich SA, Geneva, Switzerland), 9/10-ethyldiene-3-oxatricyclo[6.2.1.0(2,7)]undecane, Polywood® (perhydro-5,5,8A-trimethyl-2-naphthalenyl acetate, origin: Firmenich SA, Geneva, Switzerland), octalynol, Cetalox® (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate;
Group 5: camphor, borneol, isobornyl acetate, 8-isopropyl-6-methyl-bicyclo[2.2.2]oct-5-ene-2-carbaldehyde, camphopinene, cedramber (8-methoxy-2,6,6,8-tetramethyl-tricyclo[5.3.1.0(1,5)]undecane, origin: Firmenich SA, Geneva, Switzerland), cedrene, cedrenol, cedrol, Florex® (mixture of 9-ethylidene-3-oxatricyclo[6.2.1.0(2,7)]undecan-4-one and 10-ethylidene-3-oxatricyclo[6.2.1.0(2,7)]undecan-4-one, origin: Firmenich SA, Geneva, Switzerland), 3-methoxy-7,7-dimethyl-10-methylene-bicyclo[4.3.1]decane (origin: Firmenich SA, Geneva, Switzerland);
Group 6: Cedroxyde® (trimethyl-13-oxabicyclo-[10.1.0]-trideca-4,8-diene, origin: Firmenich SA, Geneva, Switzerland), Ambrettolide LG ((E)-9-hexadecen-16-olide, origin: Firmenich SA, Geneva, Switzerland), Habanolide® (pentadecenolide, origin: Firmenich SA, Geneva, Switzerland), muscenone (3-methyl-(4/5)-cyclopentadecenone, origin: Firmenich SA, Geneva, Switzerland), muscone (origin: Firmenich SA, Geneva, Switzerland), Exaltolide® (pentadecanolide, origin: Firmenich SA, Geneva, Switzerland), Exaltone® (cyclopentadecanone, origin: Firmenich SA, Geneva, Switzerland), (1-ethoxyethoxy)cyclododecane (origin: Firmenich SA, Geneva, Switzerland), Astrotone;
Group 7: Lilial® (origin: Givaudan SA, Vernier, Switzerland), rosinol.

By “flavor or flavoring composition”, it is meant here a flavoring ingredient or a mixture of flavoring ingredients, solvents or adjuvants of current use for the preparation of a flavoring formulation, i.e. a particular mixture of ingredients which is intended to be added to an edible composition or chewable product to impart, improve or modify its organoleptic properties, in particular its flavor and/or taste. Flavoring ingredients are well known to a person skilled in the art and their nature does not warrant a detailed description here, which in any case would not be exhaustive, the skilled flavorist being able to select them on the basis of his general knowledge and according to the intended use or application and the organoleptic effect it is desired to achieve. Many of these flavoring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, van Nostrand Co., Inc. Solvents and adjuvants of current use for the preparation of a flavoring formulation are also well known in the art.

In a particular embodiment the flavor is a mint flavor. In a more particular embodiment, the mint is selected from the group consisting of peppermint and spearmint

In a further embodiment the flavor is a cooling agent or mixtures thereof.

In another embodiment, the flavor is a menthol flavor.

Flavors that are derived from or based on fruits where citric acid is the predominant, naturally-occurring acid include but are not limited to, for example, citrus fruits (e.g., lemon, lime), limonene, strawberry, orange, and pineapple. In one embodiment, the flavors food is lemon, lime or orange juice extracted directly from the fruit. Further embodiments of the flavor comprise the juice or liquid extracted from oranges, lemons, grapefruits, key limes, citrons, clementines, mandarins, tangerines, and any other citrus fruit, or variation or hybrid thereof. In a particular embodiment, the flavor comprises a liquid extracted or distilled from oranges, lemons, grapefruits, key limes, citrons, clementines, mandarins, tangerines, any other citrus fruit or variation or hybrid thereof, pomegranates, kiwifruits, watermelons, apples, bananas, blueberries, melons, ginger, bell peppers, cucumbers, passion fruits, mangos, pears, tomatoes, and strawberries.

In a particular embodiment, the flavor comprises a composition that comprises limonene, in a particular embodiment, the composition is a citrus that further comprises limonene.

In another particular embodiment, the flavor comprises a flavor selected from the group comprising strawberry, orange, lime, tropical, berry mix, and pineapple.

The phrase flavor includes not only flavors that impart or modify the smell of foods but include taste imparting or modifying ingredients. The latter do not necessarily have a taste or smell themselves but are capable of modifying the taste that other ingredients provides, for instance, salt enhancing ingredients, sweetness enhancing ingredients, umami enhancing ingredients, bitterness blocking ingredients and so on.

The following examples are illustrative and are not meant to limit the scope of any invention claimed herein.

### EXAMPLES

### Example 1

### Microemulsions of Oil in Silk Solution (O/W Emulsions)

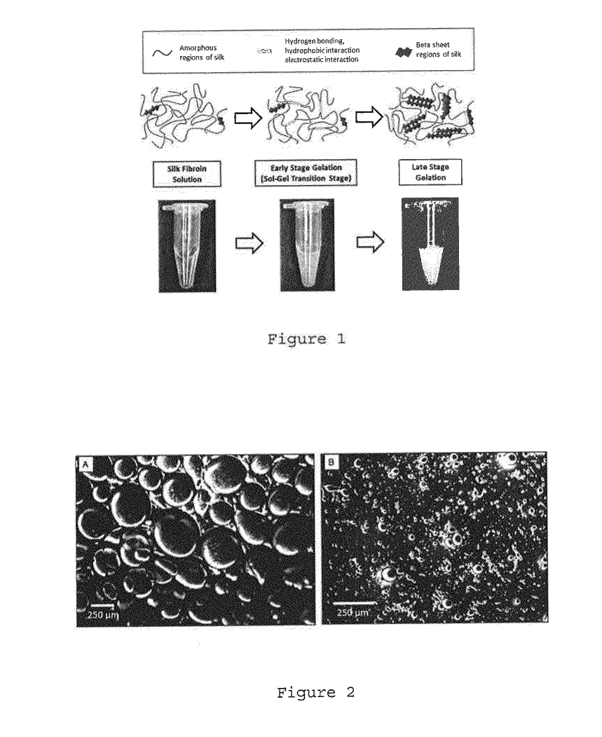

Manual mixing (gentle shaking for approx. 10 minutes) of an Oil Red O-loaded sunflower oil solution mixed with silk solution produces stable emulsions of the oil in water (O/W) type (FIG. 1A). Emulsions of sunflower oil in silk were prepared with silk concentrations of 2%, 4% and 6% (w/v) and volumetric ratios of oil to silk of 1:1, 1:2 and 1:4 and no phase separation was observed for any of the oil in silk emulsions after 48 hours stored at 4° C., compared to near total phase separation of 1:1, 1:2 and 1:4 mixtures of sunflower oil and distilled water.

Prior to sonication, an emulsion of sunflower oil containing Oil Red O mixed with 7% (w/v) aqueous silk solution in a 1:3 (v/v) ratio of oil:silk exhibited an average droplet diameter of 419.5±126.9 μm. Gentle sonication (10% amplitude for 5 seconds) of the O/W emulsions reduced the average oil particle diameter to less than 25 μm (a sample of two hundred particles in the image in FIG. 2B measured with ImageJ exhibited an average diameter of 24.6±11.4 μm, but the large number of particles less than 10 μm in diameter were not included in this average as they could not be accurately measured using ImageJ). Microemulsions prepared by sonication of sunflower oil doped with oil red O and limonene in silk are shown in FIG. 2B and FIG. 3A, respectively.

The microscale oil droplets produced by sonication are stabilized when silk protein is present in the continuous aqueous phase, and are maintained during self-assembly of silk films during drying (FIG. 3C-F) and self-assembly of silk hydrogel networks (FIG. 4B) following sonication, as expected based on the literature on proteins acting as emulsion stabilizers (Madene et al., 2006; Dickinson, 2011). Following dispersal of oil into the silk solution via sonication (FIG. 2A), this stable emulsion can be treated as silk solution and can be cast into films as previously described (Omenetto and Kaplan, 2010), rapidly-dissolving films (Kim et al., 2010), compound-loaded films for biosensors and diagnostics (Pritchard et al., 2012) and sustained release films for drug-delivery (Hofmann et al., 2006; Tsorias et al., 2012). TGA analysis revealed a slight decrease in thermostability of the silk films loaded with microparticles of oil compared with silk alone (FIG. 3B). However, self-assembly of the silk into films takes place on both Teflon coated molds (FIG. 3C-D) and patterned molds (FIG. 3E-F), even when the silk solution contained microparticles of oil. The presence of micron-scale oil droplets in the silk films renders the films opaque rather than transparent, with greater final film opaqueness resulting from higher oil content in the solution (FIG. 3C-F). Once the films were selfassembled by drying overnight at ambient conditions of temperature and pressure, re-dissolution upon exposure to distilled water and phosphate buffered saline was confirmed (data not shown). Though further evaluation of the release kinetics is needed, this suggests that if films prepared from microemulsions of oil in aqueous silk solution receive no further treatment post-drying, the silk network will re-solubilize upon exposure to aqueous media, releasing the incorporated oil microparticles. Alternately, the films can be treated by water-annealing to increase beta-sheet content in the silk network and render the films water insoluble, as has previously been described for films cast from silk alone (Jin et al., 2005).

### Example 2

### Silk Particles Produced by Drop-Wise Addition of Sonicated Silk to an Oil Bath

After confirming that microemulsions of oil are stable in aqueous silk solutions (O/W emulsion) and do not interfere with silk matrix assembly, it was next sought to evaluate a gentle, aqueous process to produce stable silk particles in oil baths, so that these two components could ultimately be integrated into O/W/O emulsions for microencapsulation. As previously described, sonication induces physical crosslinking of silk over tunable timeframes (Wang et al., 2008b). As a result of this controllable delay between the initiation of the sol-gel transition and the final onset of gelation, sonicated silk still in the solution state aliquoted into oil baths or suspended in self-stabilizing water-in-oil emulsions will complete physical crosslinking without heating or chemical treatment (unlike other emulsion-based processes for preparation of protein microspheres). Stable, physically cross-linked silk macroscale spherical particles were produced by sonicating a 6-7%, 30 minute degumming time, silk solution for approx. 30-45 seconds at an amplitude of 15%, mixing in solutions of distilled water containing model water-soluble small molecule compounds (doxorubicin or food coloring) and aliquoting the sonicated silk-drug mixture into a sunflower oil bath. In the oil bath, the aqueous silk droplets are held in a spherical conformation until gelation completes (FIG. 4C). FIG. 4A shows sonicated silk solution in the oil bath prior to the completion of gelation and FIG. 4D shows the same silk droplets after overnight incubation in the oil bath: once crosslinking of the silk network is complete, the silk droplets transition from translucent (FIG. 4A) to opaque and retain their spherical shape when removed from the oil bath (FIG. 4D). Sonication-induced microemulsion of Oil Red O loaded sunflower oil into silk prior to adding silk dropwise into the oil bath (FIG. 4B) produces cross-linked silk spherical particles with fine, microscale oil particles suspended throughout, resulting in a red coloration of the final silk macroparticle (FIG. 4E). Dehydration of physically cross-linked silk macroparticles by drying overnight at ambient conditions compresses the silk network into a smaller, dense, pellet-like particles (oil-loaded in FIG. 4F and water-soluble dye loaded in FIG. 5B).

By its nature, this extrusion-like process is characterized by precise control of particle size and compound loading due to the pipetting of controlled volumes of known composition into the oil bath. FIG. 5A shows silk hydrogel macroparticles produced by pipetting sonicated silk solution (loaded with doxorubicin post-sonication) in various volume-size droplets (from 100 μL down to 1 μL) into the sunflower oil bath. Microparticles produced by pipetting 10 μL or 50 μL of sonicated silk solution (loaded with food coloring post-sonication) and the denser, firmer, smaller particles that result when the hydrogel macroparticles are dehydrated overnight at ambient conditions are shown in FIG. 5B.

The average diameter of silk hydrogel microspheres prepared from 10 μL of sonicated silk solution loaded with dye was 2.8±0.2 mm prior to drying, and decreased to 1.9±0.3 mm after drying. The average diameter of silk hydrogel microspheres prepared from 50 μL of sonicated silk solution loaded with dye was 4.6±0.1 mm prior to during, and decreased to 2.3±0.1 mm after drying. Smaller silk microparticles (average volume less than 1 μL) were produced by dispersing silk into oil in a W/O microemulsion using sonication (FIG. 5 C-D). In some embodiments, microfluidics can be used to produce even smaller, more tightly controlled silk particles using the described approach (silk sonication followed by dropwise addition to an oil bath), as has been described for other biomaterial microparticles (Chu et al., 2007; Tan and Takeuchi, 2007; Ren et al., 2010). In addition to varying size and loading, these physically cross-linked silk particles can be further manipulated through post-crosslinking treatments: they can be (1) maintained in a rubbery, hydrated gelled state, (2) dehydrated to produce dense, hardened matrices (FIG. 4F and FIG. 5B) or (3) freeze-dried to produce dry, porous, sponge-like material (Kluge et al., 2010). These different spherical silk particles (all produced using gentle, food-safe processes) span a wide range of material properties and sizes, suitable for a diverse array of potential applications.

### Example 3

Oil-Encapsulating Silk Microparticles Derived from O/W/O Emulsions

Based on stabilization of emulsified microscale oil droplets in aqueous silk solution and sonicated silk's formation of macroscale hydrogel particles in oil baths, microparticles were prepared with a double emulsion of the type O₁/W/O₂ where O₁ this the oil of interest to encapsulate (here sunflower oil loaded with Oil Red O), W is a sonicated aqueous silk solution and O2 is a sunflower oil bath. The silk solution comprising the water phase is sonicated such that it remains in the solution phase long enough to perform the double emulsion, then completes crosslinking, thereby encapsulating the interior oil phase (schematic representation of this process shown in FIG. 6). The silk also acts as a natural emulsion stabilizer, preventing the interior oil phase (loaded with a compound of interest) from separating and leeching the compound of interest into the continuous oil phase. Morphology or O/W/O emulsions prepared from sonicated silk of varied silk composition and sonication treatment was examined with light microscopy, and diffusivity of the silk encapsulating matrices was evaluated by measuring absorbance at 518 nm of the external oil bath (an indicator Oil Red O leeching into the continuous oil phase).

O/W/O emulsions prepared with 60 minute degumming time regenerated silk fibroin solution are shown in FIG. 7. Using the higher concentration aqueous silk solution in the water phase (6% w/v) produces a dispersion of oil droplets suspended throughout the silk sphere (this encapsulation configuration is termed a microsphere, also called a matrix system (Kuang et al., 2010)) (FIG. 7A). Use of a lower concentration aqueous silk solution (3% w/v) to prepare the emulsions results in a microcapsule configuration (also called a reservoir system (Kuang et al., 2010), where one large oil droplet surrounded by a silk capsule is incorporated in each individual particle. This demonstrates that the concentration of the silk impacts the morphology of the oil encapsulating microparticle: the increased viscosity and increased protein concentration of the 6% (w/v) silk might be preventing individual droplets from coalescing into a single core droplet observed with the 3% (w/v) silk W/O/W emulsions.

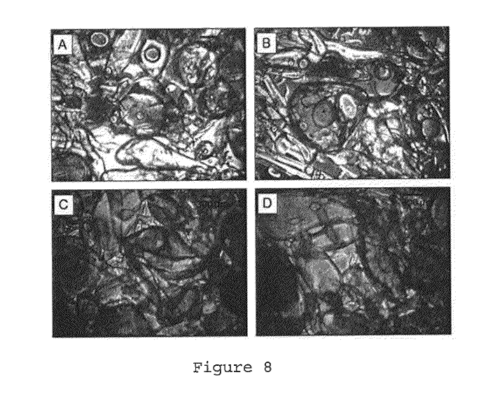

Silk solution viscosity has recently been demonstrated to decrease with increasing degumming duration, likely due to the decrease in average fragment size produced by increased exposure to heat and alkalinity (Pritchard et al., in preparation). Because increased sonication intensity accelerates the gelation process (Wang et al., 2008), increased sonication amplitude and duration are also expected to increase solution viscosity. The viscosity of the silk solution impacts particle morphology and silk's permeability as an encapsulant material. Representative images of W/O/W emulsions produced using 6% (w/v) silk prepared using a 30 minute degumming time are shown in FIG. 8. Compared with the lower viscosity, shorter fragment size 60 minute degumming time silk emulsions, the particles are less spherical and oil encapsulation appears less regular. When sonication intensity increases (10% for 15 seconds in FIG. 8A-B, compared to 15% for 15 seconds in FIG. 8C-D), resulting particles are even more elongated and irregular. The shorter degumming time combined with the increased sonication intensity may cause premature crosslinking, preventing the silk in the emulsion from incorporating an interior oil droplet or adopting a spherical conformation.

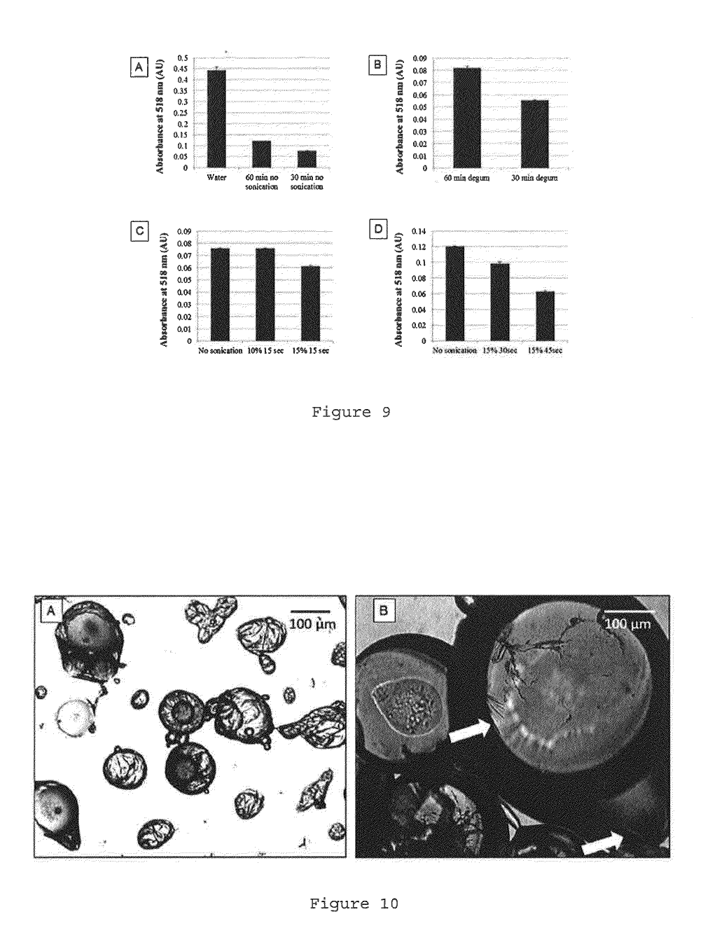

During the preparation of microcapsules, material composition and diffusivity of the encapsulating matrix material determine the retention degree of core compounds (Gharsallaoui et al., 2007). At higher solution viscosities, absorbance at 518 nm (an indicator of the Oil Red O content) of the external oil phase (i.e. the sunflower oil bath) decreases, suggesting the permeability of the silk capsule to the Oil Red O in the internal oil phase and consequent “loss” of compound loaded in the internal phase decreases as the viscosity of the silk solution in the double emulsion increases. Compared with an aqueous phase of plain distilled water, unsonicated silk also reduces loss to the external oil phase (FIG. 9A). When silk concentration is held constant and sonication treatment is held constant, Oil Red O loss to the external phase decreases with decreasing degumming time/increasing silk solution viscosity (FIG. 9B). Similarly, when silk solution concentration and degumming time are held constant (6% (w/v), 30 minute degumming time in FIG. 9C; 6% (w/v), 60 minute degumming time in FIG. 9D), and sonication intensity increases (amplitude or duration or both), Oil Red O loss generally decreases (with the exception of 6% (w/v) 30 minute degumming time silk exhibiting no change in Oil Red O loss for unsonicated silk solution compared with silk solution sonicated for 15 seconds at an amplitude of 15%, possibly because this sonication treatment does not significantly increase viscosity).

The sunflower oil bath comprising the continuous, external oil phase in O/W/O emulsions prepared with distilled water containing no silk as the water phase exhibited the highest absorbance at 518 nm (0.442±0.014), indicating the greatest loss of Oil Red O from the internal oil capsule into the continuous oil phase. The continuous oil phases in O/W/O emulsions with unsonicated aqueous silk fibroin solution prepared using a 60 minute and 30 minute degumming time as the water phase had absorbance values at 518 nm of 0.12±0.001 and 0.076±0.001, respectively. The presence of silk in the water phase reduces Oil Red O leeching into the oil phase, and the increase in viscosity/average fragment length of the silk solution prepared using the shorter degumming time further increases compound retention in the interior oil core (FIG. 10A). In addition to silk processing parameters, Oil Red O retention in the interior oil core is also controlled by sonication treatment and concentration (w/v) of the silk solution in the water phase (FIG. 10B-D, Table 2). In addition to the observation that silk encapsulation provides a barrier to Oil Red O diffusion into the external oil phase, morphology of the silk O/W/O emulsions suggests that the silk in the aqueous layer assembles into a capsule around the interior oil phase: puckering and wrinkling of the silk “skin” are apparent (FIG. 10).

TABLE 2
Absorbance at 518 nm of external oil phase in O/W/O emulsions with
water phases comprised of aqueous silk solutions with varied properties
(degumming treatment duration and concentration)
exposed to varied sonication treatment (treatment duration and amplitude).

| Silk Properties | | | | Absorbance at 518 nm |
|---|---|---|---|---|
| Degumming | Silk Concentration | Sonication Treatment | | of external oil phase |
| Duration (min) | (w/v) | Amplitude | Duration (sec) | (sunflower oil bath) |
| 60 | 6% | None | None | 0.12 ± 0.001 |
| | 6% | 15% | 30 | 0.098 ± 0.003 |
| | 6% | 15% | 45 | 0.063 ± 0.002 |
| | 3% | 15% | 30 | 0.082 ± 0.002 |
| 30 | 6% | None | None | 0.076 ± 0.001 |
| | 6% | 10% | 15 | 0.076 ± 0.001 |
| | 6% | 15% | 15 | 0.061 ± 0.001 |
| | 3% | 15% | 30 | 0.055 ± 0.001 |
| | 3% | 15% | 15 | 0.072 ± 0.016 |

### Materials and Methods

### Materials

Cocoons of Bombyx mori silkworm silk were purchased from Tajima Shoji Co., LTD (Sumiyoshicho, Naka-ku, Yokohama, Japan). Sunflower oil, doxorubicin and Oil Red O were purchased from Sigma Aldrich (St. Louis, Mo.). Limonene was provided by Firmenich (Newark, N.J.).

### Silk Solution and Materials Preparation

Silk fibroin solution was prepared from B. mori cocoons as previously described (Sofia et al., 2001). Briefly, cocoons were boiled for either 30 min or 60 min in a solution of 0.02 M Na₂CO₃ and rinsed, then dried at ambient conditions overnight. The dried fibroin was solubilized in a 9.3 M aqueous LiBr solution at 60° C. for 2-4 h, yielding a 20% (w/v) solution. LiBr was then removed from the silk by dialyzing the solution against distilled water for 2.5 days using Slide-a-Lyzer dialysis cassettes (MWCO 3,500, Pierce Thermo Scientific Inc., Rockford, Ill.). Silk fibroin concentration was determined by evaporating water from a solution sample of known volume and massing using an analytical balance. Silk solutions were stored at 4-7° C. before use.

### Silk Film Casting

Silk films were cast as previously described (Hofmann et al., 2006). Briefly, silk solution was aliquoted into Teflon coated molds or patterned molds, then dried overnight at ambient conditions. Oil-loaded silk films were prepared by sonicating oil into silk solution of the desired concentration at various volumetric ratios of oil:silk using a Branson Digital Sonifier 450 at 10-15% amplitude for 5 seconds, then aliquoting and casting as described.

### Sonication-Induced Silk Gelation

Sonication-induced gelation was carried out as previously described (Wang et al., 2008b). Briefly, silk solution of the desired concentration and prepared with the degumming duration of interest was sonicated using a Branson Digital Sonifier 450 at 10-15% amplitude for varied duration (the various conditions of silk concentration, degumming duration and sonication amplitude and duration are specified throughout the results section). Emulsions were prepared with sonicated or unsonicated silk as described throughout the results section.

### Thermogravimetric Analysis

Thermogravimetric analysis (TGA) (TA Instruments Q500) was used to measure weight changes of silk films assembled from 1% w/v silk fibroin solutions. TGA curves were obtained under nitrogen atmosphere with a gas flow of 50 mL/min. Analysis was first performed by heating the sample from 25° C. to 600° C. at a rate of 2° C./min. Silk film weight loss was recorded as a function of temperature.

### REFERENCES I

Altman G H, Horan R L, Lu H, Moreau J, Martin I, Richmond J C, Kaplan D L. Silk matrix for tissue engineered anterior cruciate ligaments. Biomaterials 2002; 23:4131-4141.
Arshady R. Albumin microspheres and microcapsules: methodology of manufacturing techniques. J Control Release 1990; 14:111-131.
Baranauskienė R, Venskutonis P R, Dewettinck K, Verhé R. Properties of oregano (Origanum vulgare L.), citronella (Cymbopogon nardus G.) and marjoram (Majorana hortensis L.) flavors encapsulated into milk protein-based matrices. Food Research International. 2006; 39:413-425.
Bayraktar O, Malay O, Özgarip Y, Batigün A. Silk fibroin as a novel coating material for controlled release of theophylline. Eur J Pharm Biopharm 2005; 60:373-381.
Berthier D L, Schmidt I, Fieber W, Schatz C, Furrer A, Wong K, Lecommandoux S. Controlled release of volatile fragrance molecules from PEO-b-PPO-b-PEO block copolymer micelles in ethanol-water mixtures. Langmuir 2010; 26:7953-7961.
Chourpa I, Ducel V, Richard J, Dubois P, Boury F. Conformational modifications of a gliadin and globulin proteins upon complex coacervates formation with gum Arabic as studied by Raman microspectroscopy. Biomacromolecules 2006; 7:2616-2623.
Chu L-Y, Utada A S, Shah R K, Kim J-W, Weitz D A. Controllable monodisperse multiple emulsions. Angew Chem Int Ed 2007; 46:8970-8974.
Desobry S A, Netto F M, Labuza T P. Comparison of spray-drying, drum-drying and freeze-drying for β-carotene encapsulation and preservation. Journal of Food Science 1997; 62:1158-1162.
Diab T, Pritchard E M, Uhrig B E, Boerckel J D, Kaplan D L, Guldberg R E. A silk hydrogel-based delivery system of bone morphogenetic protein for the treatment of large bone defects. J Mech Behav Biomed Mater. In press
Dickinson E. Double emulsions stabilized by food biopolymers. Food Biophysics 2011; 6:1-11.
Edris A, Bergnståhl B. Encapsulation of orange oil in a spray dried double emulsion. Nahrung 2001; 45:133-137.
Elzoghby A O, Samy W M, Elgindyoghby N A, Samy W M, Elgindy N A. Albumin-based nanoparticles as potential controlled release drug delivery systems. J Control Release 2012; 157:168-182.
Esposito E, Cortesi R, Nastruzzi C. Gelatin microspheres: influence of preparation parameters and thermal treatment on chemico-physical and biopharmaceutical properties. Biomaterials 1996; 17:2009-2020.
Gibbs BF, Kermasha S, Alli I, Mulligan C N. Encapsulation in the food industry: a review. Int J Food Sci Nutr 1999; 50:213-224.
Gharsallaoui A, Roudaut G, Chambin O, Voilley A, Saurel R. Applications of spray-drying in microencapsulation of food ingredients: An overview. Food Research International 2007; 40:1107-1121.
Gouin S. Microencapsulation: industrial appraisal of existing technologies and trends. Trends in Food Sci Technol 2004; 15:330-347.
Hanawa T, Watanabe A, Tsuchiya T, Ikoma R, Hidaka M, Sugihara M. New oral dosage form for elderly patients. II. release behavior of benfotiamine from silk fibroin gel. Chem Pharm Bull 1995; 43:872-876.
Hofmann S, Wong Po Foo C T, Rossetti F, Textor M, Vunjak-Novakovic G, Kaplan D L, Merkle H P, Meinel L. Silk fibroin as an organic polymer for controlled drug delivery. J Control Release 2006; 111: 219-227.
Horan R L, Antle K, Collette A L, Wang Y, Huang J, Moreau J E, Volloch V, Kaplan D L, Altman G H. In vitro degradation of silk fibroin. Biomaterials 2005; 26:3385-3393
Imsombut T, Srisuwan Y, Srihanam P, Baimark Y. Genipin-cross-linked silk fibroin microspheres prepared by the simple water-in-oil emulsion solvent diffusion method. Powder Technol 2010; 203:603-608.
Ishii A, Furukawa M, Matsushima A, Kodera Y, Yamada A, Kanai H, Inada Y. Alteration of properties of natural pigments by conjugation with fibroin or polyethylene glycol. Dyes and Pigments 1995; 27:211-217.
Jayakrishnan A, Knepp W A, Goldberg E P. Casein microspheres: preparation and evaluation as a carrier for controlled drug delivery. International Journal of Pharmaceutics 1994; 106:221-228.
Jin H-J, Park J, Karageorgiou V, Kim U J, Valluzzi R, Cebe P, Kaplan D L. Water-insoluble silk films with reduced (3-sheet content. Adv Funct Mater 2005; 15:1241-1247.
Jun-xia X, Hai-yan Y, Jian Y. Microencapsulation of sweet orange oil by complex coacervation with soybean protein isolate/gum Arabic. Food Chemistry 2011; 125:1267-1272.
Kanakdande D, Bhosale R, Singhal R S. Stability of cumin oleoresin microencapsulated in different combination of gum arabic, maltodextrin and modified starch. Carbohydrate Polymers 2007; 67:536-541.
Kim D-H, Viventi J, Amsden J J, Xiao J, Vigeland L, Kim Y-S, Blanco J A, Panilaitis B, Frechette E S, Contreras D, Kaplan D L, Omenetto F G, Huang Y, Hwang K-C, Zakin M R, Litt B, Rogers J A. Dissolvable films of silk fibroin for ultrathin, conformal biointegrated electronics. Nat Mater 2010; 9:511-517.
Kluge J A, Rosiello N C, Leisk G G, Kaplan D L, Dorfmann A L. The consolidation behavior of silk hydrogels. J Mech Behav Biomed Mater 2010; 3:278-89.
Kuang S S, Oliveira J C, Crean A M. Microencapsulation as a tool for incorporating bioactive ingredients into food. Crit Rev Food Sci Nutr 2010; 50:951-968.
Leal-Egaña A, Scheibel T. Silk-based materials for biomedical applications. Biotechnol Appl Biochem 2010; 55:155-167.
Madene A, Jacquot M, Scher J, Desobry S. Flavour encapsulation and controlled release—a review. Int J Food Sci Technol 2006; 41:1-21.
Matsumoto A, Chen J, Collette A L, Kim U-J, Altman G H, Cebe P, Kaplan DL. Mechanisms of silk fibroin sol-gel transitions. J Phys Chem B 2006; 110:21630-21638.
Meinel L, Hofmann S, Karageorgiou V, Kirker-Head C, McCool J, Gronowicz G, Zichner L, Langer R, Vunjak-Novakovic, Kaplan D L. The inflammatory responses of silk films in vitro and in vivo. Biomaterials 2005; 26:147-155.
Numata K, Kaplan D L. Silk-based delivery systems of bioactive molecules. Adv Drug Deliv Rev. 2010; 62:1497-1508.
Omenetto F G, Kaplan D L. New opportunities for an ancient material. Science 2010; 329:528-531. Ouali L, Léon G, Normand V, Johnsen H, Dyrli A, Schmid R, Benczedi D. Mechanism of Romascone® release from hydrolyzed vinyl acetate nanoparticles: thermogravimetric method. Polym Adv Technol 2006; 17:45-52.
Panilaitis B, Altman G, Chen J, Jin H J, Karageorgiou V, Kaplan, D L. Macrophage responses to silk. Biomaterials 2003; 24:3079-3085.
Pritchard E M, Kaplan, D L. Silk fibroin biomaterials for controlled release drug delivery. Expert Opin Drug Deliv 2011; 8:1-15.
Pritchard E M, Dennis P B, Omenetto F, Naik R R, Kaplan D L. Physical and chemical aspects of stabilization of compounds in silk. Biopolymers in press
Qian J, Liu Y, Liu H, Yu T, Deng J. An amperometric new methylene blue N-mediating sensor for hydrogen peroxide based on regenerated silk fibroin as an immobilization matrix for peroxidase. Anal Biochem 1996; 236:208-214.
Ren P-W, Ju X-J, Xie R, Chu L-Y. Monodisperse alginate microcapsules with oil core generated from a microfluidic device. Journal of Colloid and Interface Science 2010; 343:392-395.
Sofia S, McCarthy M B, Gronowicz G, Kaplan D L. Functionalized silk-based biomaterials for bone formation. J Biomed Mater Res 2001; 54:139-148.
Sohail A, Turner M S, Coombes A, Bostrom T, Bhandari B. Survivability of probiotics encapsulated in alginate gel microbeads using a novel impinging aerosols method. Int J Food Microbiol 2011; 145:162-168.
Tan W-H, Takeuchi S. Monodisperse alginate hydrogel microbeads for cell encapsulation. Adv Mater 2007; 19:2696-2701.
Wang X, Kluge J A, Leisk G G, Kaplan D L. Sonication-induced gelation of silk fibroin for cell encapsulation. Biomaterials 2008b; 29:1054-1064.
Wang Y, Rudym D D, Walsh A, Abrahamsen L, Kim H J, Kim H S. In vivo degradation of three-dimensional silk fibroin scaffolds. Biomaterials 2008a; 29: 3415-3428. Wang Y, Kim H-J, Vunjak-Novakovic G, Kaplan D L. Stem cell-based tissue engineering with silk biomaterials. Biomaterials 2006; 27:6064-6082.
Wenk E, Merkle H P, Meinel L. Silk fibroin as a vehicle for drug delivery applications. J Control Release 2011; 150:128-141.
Wray L S, Hu X, Schmidt D, Kaplan D L. Effect of processing on silk-based biomaterials: reproducibility and biocompatibility. J Biomed Mater Res B Appl Biomater 2011; 99:89-101.
Zhang X, Reagan M R, Kaplan D L. Electrospun silk biomaterial scaffolds for regenerative medicine. Adv Drug Deliv Rev 2009; 61:988-1006.

### REFERENCES II

D.-H. Kim, J. Viventi, J. J. Ams J. Xiao, L. Vigeland, Y.-S. Kim, J. A. Blanco, B. Panilaitis, E. S. Frechette, D. Contreras, D. L. Kaplan, F. G. Omenetto, Y. Huang, K.-C. Hwang, M. R. Zakin, B. Litt, J. A. Rogers. Dissolvable films of silk fibroin for ultrathin conformal biointegrated electronics. Nature Mater. 2010, 9, 511-517.
Xiaoqin Wang, Jonathan A. Kluge, Gary G. Leisk, David L. Kaplan. Sonication-induced gelation of silk fibroin for cell encapsulation. Biomaterials 29 (2008) 1054-1064

1. An oil in water emulsion comprising water, silk fibroin and an oil wherein the emulsion comprises a droplet comprising the oil encapsulated by the silk fibroin.

2. The emulsion as recited in claim 1 wherein the concentration of the silk fibroin is provided in an amount of about 6% (w/v) of the emulsion.

3. The emulsion as recited in claim 2 wherein the amount of the oil provided is in a ratio to the amount of silk fibroin provided of about 1:1 (v/v).

4. The emulsion as recited in any one of claims 1-3 wherein the oil comprises a flavor or fragrance.

5. The emulsion as recited in claim 1-4 where the droplet has an average diameter of form about 2 micrometers up to about 1,000 micrometers

6. The emulsion as recited in any one of claim 5 wherein the droplet has an average diameter of up to about 130 micrometers.

7. The emulsion as recited in claim 6 wherein the droplet has an average diameter of up to about 25 micrometers.

8-26. (canceled)

27. A particle comprising a physically cross-linked silk fibroin and an oil.

28. The particle as recited in claim 27 wherein the particle comprises a microsphere.

29. The particle as recited in claim 28 wherein the amount of the oil is provided in an amount of about 10 to about 99%, by weight, of the total weight of the particle.

30. The particle as recited in claim 29 wherein the amount of the oil is provided in an amount of about 50%, by weight, of the total weight of the particle.

31. The particle as recited in claim 27 wherein the particle comprises a microcapsule.

32-109. (canceled)

110. A silk particle comprising at least two immiscible phases, a first immiscible phase comprising a silk-based material and a second immiscible phase comprising an active agent, wherein the first immiscible phase encapsulates the second immiscible phase and the second immiscible phase excludes a liposome.

111. The silk particle of claim 110, wherein the second immiscible phase comprises a lipid component.

112. The silk particle of claim 111, wherein the lipid component comprises oil.

113. The silk particle of claim 110, wherein the second immiscible phase forms a single compartment.

114. The silk particle of claim 110, wherein the second immiscible phase forms a plurality of compartments.

115. The silk particle of claim 113, wherein the size of the compartment or compartments ranges from about 1 μm to about 1000 μm, or from about 10 μm to about 500 μm.

116. The silk particle of claim 110, wherein the active agent present in the second immiscible phase comprises a hydrophobic or lipophilic molecule.

117-194. (canceled)

195. The silk particle of claim 114, wherein the size of the compartment or compartments ranges from about 1 μm to about 1000 μm, or from about 10 μm to about 500 μm.
